(19) United States
(12) Patent Application Publication
(10) Pub. No.: US 2003/0093830 A1
(43) Pub. Date: May 15, 2003

(54) Means for identifying the locus of a major resistance gene to the rice yellow mottle virus, and their applications

(76) Inventors: Alain Ghesquiere, Montpellier (FR); Laurence Albar, Mons (FR)

Correspondence Address: NIXON & VANDERHYE P.C., 8th Floor, 1100 North Glebe Road, Arlington, VA, 22201-4714, US

(21) Appl. No.: 10/023,476

(22) Filed: Dec. 20, 2001

Related U.S. Application Data
(63) Continuation-in-part of application No. PCT/FR00/01742, filed on Jun. 21, 2000.

(30) Foreign Application Priority Data
Jun. 21, 1999 (FR) 99/07834

(51) Int. Cl. A01H 5/00; C12Q 1/68

(52) U.S. Cl. 800/279; 800/320.2; 435/6

(57) ABSTRACT

The invention relates to a method for identifying the markers of the locus of a major resistance gene with respect to RYMV. The invention method comprises the following steps: selective amplification of fragments of rice DNA from resistant individuals and sensitive individuals descending from parental varieties, whereby said fragments undergo a prior digestion phase followed by ligation in order to fix additional initiators having one or more specific nucleotides at the extremities thereof; separation of the amplification products; comparison of the electrophoresis profiles obtained by mixing fragments from resistant descendants and sensitive de4scendants with fragments from parental varieties, in order to identify strips where polymorphism is genetically linked to the resistance locus. Said identification is followed by a validation step in which verification occurs on all individuals and the genetic recombination rate between the marker and the resistance locus is calculated. The invention can be used to identify resistant phenotypes and transfer the RYMV resistance gene.

### This application is a continuation-in-part of PCT Application No. PCT/FROO/01742, filed Jun. 21, 2000, which designated the U.S., the entire content of which is incorporated herein by reference.

The invention relates to the means, tools and methods for identifying the locus of a major resistance gene to the Rice Yellow Mottle Virus (RYMV) in short. It more particularly relates to markers and PCR primers in respect of tools.

RYMV is a virus that is endemic in Africa. In a few rare varieties of the African species of cultivated rice Oryza glaberrima, a very high resistance to RYMV has been identified. But since the interspecific hybrids between the two species of cultivated rice are extremely sterile, prior research has not been able to describe either the genetic bases or the mechanism of this resistance.

Research by the inventors in this area has shown that a variety called Gigante which originated from Mozambique and was identified by ADRAO, and which is a member of the cultivated Asian rice species Oryza sativa, shows the same characteristics as those observed with O. glaberrima. The inventors have characterized RYMV resistance by demonstrating that it is related to a major recessive resistance gene that is identical in both sources of resistance under consideration (O. Sativaand O. glaberrima).

This resistance occurs at the level of cell-to-cell movement and leads to blockage of the virus at the infected cells whereas virus replication is normal.

On the contrary, in resistant varieties, a mutation of said protein does not enable anymore the association with the virus, and thus its diffusion in the plant.

The migration of RYMV occurs under the form of a mucleoproteic complex associating viral nucleic acid, of protein and movement virus protein. In the cells of sensitive varieties, an host factor, profably a protein, also contributes to the movement of the virus.

Having regard to these results, the inventors prepared specific method and tools for characterizing the chromisome fragment bearing said resistance to RYMV gene which codes for said protein enabling the movement of the virus in the plant.

The purpose of the invention is therefore to provide a method for identifying molecular markers of the resistance locus to RYMV.

It also concerns the DNA fragments such as revealed by this method and which can be used as markers.

The invention also concerns applications of such markers, in particular to define other markers having high specificity to the resistance locus and to predict a resistant phenotype.

The invention further relates to sequences of primers, as new products, used in the PCR techniques applied.

According to the invention, the identification of markers of the locus of a major resistance gene to RYMV, comprises the use of AFLP markers (Amplified Fragments Length Polymorphism) and uses the PCR technique.

This method of identification is characterized in that it comprises:

selective amplification of rice DNA fragments firstly from resistant individuals and secondly from sensitive individuals, descending from parent varieties, these fragments being previously submitted to a digestion step, followed by ligation to fix complementary primer adapters having, at their end, one or more specific nucleotides, one of the primers in the primer pair being labelled for development purposes,

separating the amplification products by gel electrophoresis under denaturing conditions, and

comparing the electrophoresis profiles obtained with mixtures of fragments derived from resistant descendants and with mixtures derived from sensitive descendants, with the fragments derived from parent varieties, for the purpose of identifying bands whose polymorphism is genetically linked to the resistance locus, this identification being optionally followed, for validation purposes, by verification on each of the individuals and by calculation of the genetic recombination rate between the marker and the resistance locus.

In one embodiment of the invention, the DNA fragments are obtained by digestion of the genomic DNAs of resistant plants and of sensitive plants, and their parents, using restriction enzymes.

Restriction enzymes which have proved to be suitable include EcoRI and MseI.

Short nucleotide sequences are fixed to digestion fragments (adapters) to generate blunt ends to which the adapters are subsequently fixed.

The primers used in the amplification step are complementary to these adapters with, at their 3′ end, from 1 to 3 nucleotides which may be variable.

The amplification step is advantageously conducted using the PCR technique.

Specific amplification profiles are obtained with primer pairs respectively having AAC and CAG, ACC and CAG motifs at their end, or further AGC and CAG.

The sequences corresponding to the EcoRI and MSeI adapters are respectively GAC TGC GTA CCA ATT C (SEQ ID N^{o} 1) and GAT GAG TCC TGA GTA A (SEQ N^{o} 2).

The primer pairs used for amplification are then advantageously chosen from among E-AAC/M-CAG; E-ACC/M-CAG; and E-AGC/M-CAG; in which E and M respectively correspond to SEQ ID N^{o} 1 and SEQ ID N^{o} 2. Other pairs are given in table 6 in the examples.

Comparative study of the amplification profiles obtained reveals polymorphic bands specifically present in the sensitive varieties and their sensitive descendants, as shown in the examples, and consequently corresponding to resistance markers.

In particular, development by gel electrophoresis under denaturing conditions leads to identifying 2 marker bands M1 and M2 of respectively 510 bp and 140 bp.

According to analysis of segregation data, these 2 bands determine a chromosome segment of 10 to 15 cM carrying the resistance locus and are located either side of this locus at 5-10 cM.

According to one provision of the method of the invention, the polymorphic bands identified as markers specific to the RYMV resistance locus, are isolated from gels. Advantageously the electrophoresis gels are excised. This isolation step is followed by purification using conventional techniques. In this manner DNA fragments are obtained.

According to another provision of the invention, said purified fragments are cloned in an appropriate vector, such as a plasmid, inserted into the host cells, in particular bacterial cells such as those of E. coli.

According to another provision of the invention, the purified, cloned DNA fragments are sequenced.

Taking advantage of the sequences of the inserts corresponding to said DNA fragments, the invention also provides a method for obtaining markers having high specificity for the locus of a major resistance gene to RYMV. This method is characterized in that PCR primer pairs are determined which are complementary to the fragments of the sequence of a given insert, specific amplification of the insert is made using these primer pairs, and the amplification products are then subjected to migration on electrophoresis gel.

These DNA sequences can be used to identify a polymorphism linked to the resistance locus in a rice variety to be examined using different methods as described in the examples:

1) by directly identifying a size polymorphism of these DNA sequences after specific amplification and separation of the fragments on agarose gel,

2) by digesting the amplification products with restriction enzymes to separate the digestion products on agarose gel,

3) by using these sequences as probes to hybridize the DNA of rice varieties previously digested by a restriction enzyme and to determine a restriction polymorphism.

The invention concerns, as new products, the polymorphous AFLP bands such as identified by the method defined above, from the DNA of rice plants, optionally isolated, purified and sequenced.

These AFLP bands are characterized in that they are specifically revealed in a variety sensitive to RYMV (IR64) and in the fraction of sensitive plants derived from the crossing of this variety with the Gigante resistance variety as described in the examples.

The invention particularly concerns the DNA sequences corresponding to these polymorphous bands, which can be used to define a segment of chromosome 4 of 10-15 cM carrying the resistance locus to RYMV.

Having regard to the method with which they are obtained, the AFLP bands correspond to restriction fragments and in particular, according to one embodiment of the method of the invention, to EcoRI-MseI fragments.

Fragments of this type are called M1 and M2 markers and are characterized by a size, of 510 bp and 140 bp respectively, in electrophoresis gel under denaturing conditions.

These fragments are characterized in that they correspond to DNA sequences flanking the resistance locus and located either side of the latter at 5-10 cM.

The invention also concerns fragments cloned in vectors such as plasmids, these cloning vectors as such, characterized in that they comprise such fragments, and the host cells transformed using these vectors, such as bacterial cells, for example E. coli.

The invention relates in particular to the DNA sequence corresponding to the fragment identified as M1 marker and meeting the following sequence SEQ ID N^{o} 3:

CGTGCTTGCTTATAGCACTACAGGAGAAGGAAGGGGAACACAACAGC
CATGGCGAGCGAAGGTTCAACGTCGGAGAAACAGGCTGCGACGGGCA
GCAAGGTGCCGGCGGCGGATCGGAGGAAGGAAAAGGAGGAAATCGA
AGTTATGCTGGAGGGGCTTGACCTAAGGGCAGATGACGAGGAGGATG
TGGAATTGGAGGAAGATCTAGAGGAGCTTGAGGCAGATGCAAGATGG
CTAGCCCTAGCAACAGTTCATACGAAGCGATCGTTTAGTCAAGGGGCT
TTCTTTGCGAGTATGCGCTCAGCATGGAACTGCGCGAAAGAAGTAGAT
TTCAGAGCAATGAAAGACAATCTGTTCTCGATCCAATTCAATTGTTTG
GGGGATTGGGAACGAGTTATGAATGAAGGTCCATGGACCTTTCGAGG
ATGTTCGGTGCTCCTCGCAGAATATGATGGCTGGTCCAAGATTGAAT

The DNA sequence of the Ml marker has a size of 471 bp.

The invention also concerns, as new products, the sequences of nucleotides used as PCR amplification primers.

Such primers comprise the pairs E-AAC/M-CAG; E-ACC/M-CAG; E-ACC/M-CAG; in which E and M respectively relate to SEQ ID N^{o} 1 and SEQ ID N^{o} 2.

Other primers are complementary to sequences identified in the sequence of the fragment designated by marker M1. These are in particular (5 ′,3′) sequences chosen from among:

| | | |
|---|---|---|
| AGGAAGGGGAACACAACAGCC | (21 bp) | (SEQ ID NO 4) |
| TTATGCTGGAGGGGCTTGACC | (21 bp) | (SEQ ID NO 5) |
| GCAGTTCCATGCTGAGCGCAT | (21 bp) | (SEQ ID NO 6) |
| CCGAACATCCTCGAAAGGTCC | (21 bp) | (SEQ ID NO 6) |
| TCATATTCTGCGAGGAGCACC | (21 bp) | (SEQ ID NO 8) |

The invention also concerns the DNA sequence corresponding to the fragment identified as marker M2 and corresponding to sequence SEQ ID N^{o} 9

AATTCACCCC ATGCCCTAAG TTAGGACGTT CTCAGCTTAG
TGGTGTGGTA GCTTTTTCTA TTTTCCTAAG CACCCATTGA
AGTATTTTGC ATTGGAGGTG GCCTTAGGTT TGCCTCTGTTA

The size of M2 is 120 bp.

Specific primers complementary to sequences identified in the sequence of M2 were defined. Said sequences meet the following sequencing (5′,3′):

| | |
|---|---|
| AACCTAAGGCCACCTCCAAT | SEQ ID NO 10 |
| GCAAACCTAAGGCCACCTC | SEQ ID NO 11 |
| ATTCACCCCATGCCCTAAG | SEQ ID NO 12 |

According to a further aspect of the invention, the latter concerns the use of DNA sequences obtained with the above primers to define polymorphisms which can be used to identify resistant phenotypes.

The invention also concerns a method for identifying the DNA sequence carrying the major resistance gene to RYMV. This method is characterized by screening a bank consisting of DNA fragments of 100 to 150 kb of the IR64 or other variety, such as the BAC bank (Bacterial Artificial Chromosomes) cloned in bacteria, to select the clone or clones from the bank containing the markers defined above and the resistance gene to RYMV.

This type of BAC bank is available from the IRRI institute.

The existence of different restriction sites on the sequence corresponding to the M1 marker, and in particular the sites corresponding to HpaII/Mspl, provides for advantageous identification of resistant phenotypes.

The identification of different restriction sites on the sequence corresponding to the M1 marker enables characterization of a polymorphism which may be put to advantageous use to map the M1 marker on rice genetic linkage maps.

The map of the sequence corresponding to the M1 marker can be used to identify a chromosomal zone on chromosome 4 of rice carrying the RYMV resistance locus.

The map of the RYMV resistance gene on chromosome 4 of the rice genetic map allows identification of the markers the closest to the resistance locus. These are in particular the microsatellite markers RM252 and RM273 or any other marker inside the (4-5cM) space defined by these markers allowing identification of a polymorphism between the IR64 and Gigante parents, such as the RFLP markers screened from genomic banks or cDNA, microsatellites, AFLP markers or markers derived from physical mapping of the region such as BAC, YAC clones or their cosmids.

The markers identified in accordance with the invention, or any other marker located in this space allowing identification of a polymorphism between resistant varieties such as Gigante or O. Glaberrima with RYMV-sensitive rice varieties, may be used for transfer of RYMV resistance into sensitive varieties by successive backcrosses followed by marker-assisted selection.

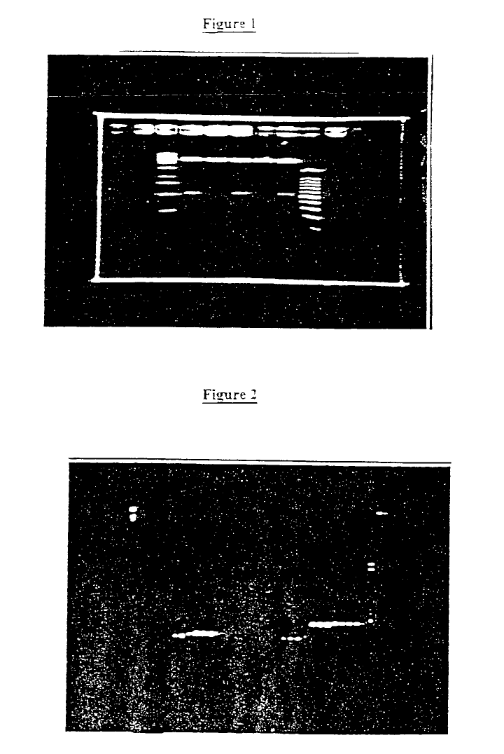

Other characteristics and advantages of the invention will be given in the following examples, in which reference is made to FIGS. 1 to 10 which respectively represent:

FIG. 1: cloning of marker M1 in the PGEMTeasy plasmid. Digestion of the plasmid shows a DNA fragment of 510 bp corresponding to band M1;

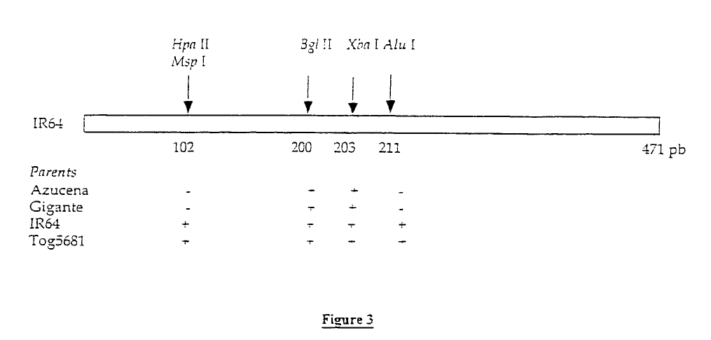

FIG. 2: amplification of marker Ml in the four rice varieties (Azucena, Gigante, IR64 and Tog5681) using the primer pairs (2-4): 291 bp; (2-5): 310 bp; (1-3): 288 bp; (1-4): 406 bp; (1-5): 425 bp; (2-3). The M1 fragment is slightly bigger in Tog5681 than in the other varieties;

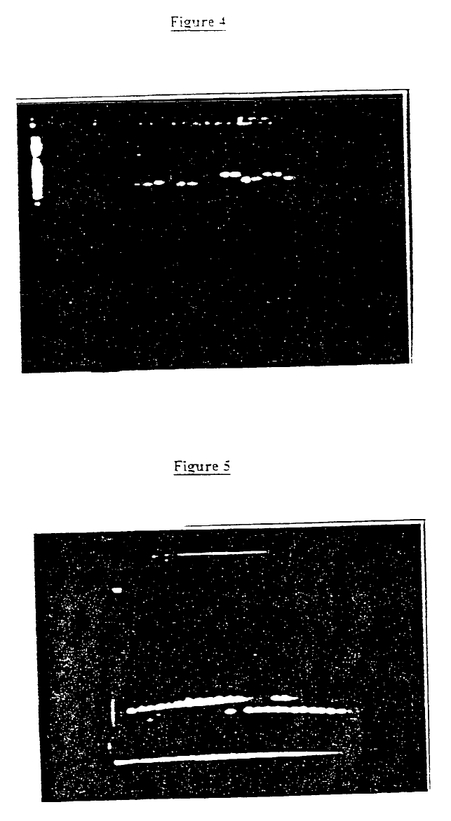

FIG. 3: identification of restriction sites on the sequence of the M1 marker in the 4 varieties IR64, Azucena, Gigante and Tog5681;

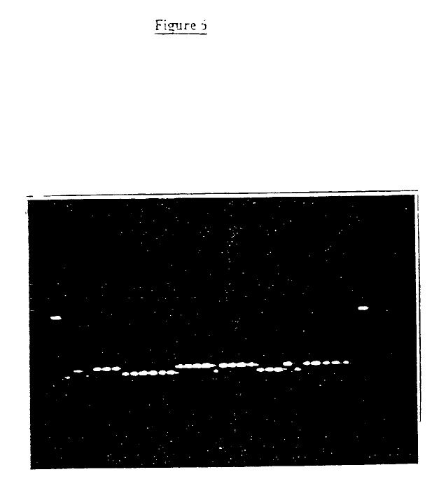

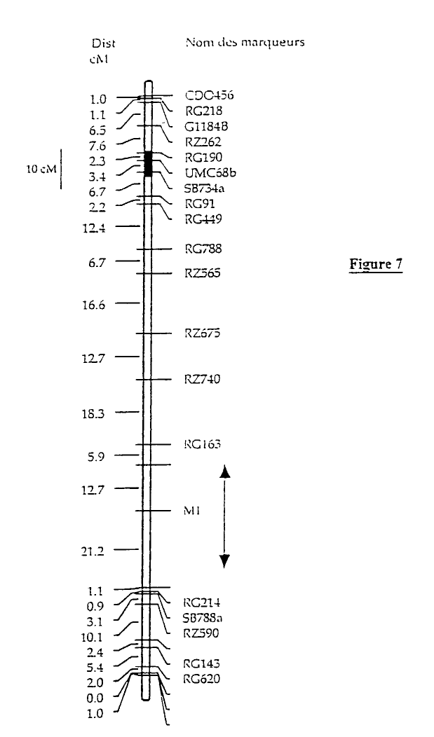

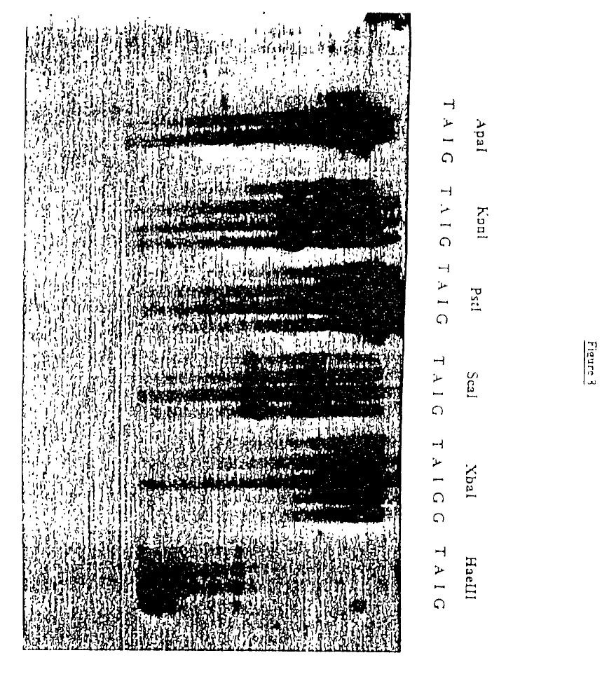

FIG. 4: digestion of the Ml marker with the HpaII enzyme after PCR amplification using primer pairs (1-3), (1-4) and (1-5)on the four varieties (Azucena, Gigante, IR64 and Tog5681). The presence of a HpaII restriction site in the IR64 and Tog568 varieties releases a fragment of 86 bp which reduces the size of the amplified fragment to the same extent.

FIG. 5: characterization of the M1 marker on sensitive and resistant plants of F2 issue(IR64 and Gigante). The resistant F2 plants have the profile of the resistant parent (IR64 - no Hpall site), with the exception of a single recombinant, the resistant plants have the profile of the sensitive parent (IR64-presence of HpaII site) with the exception of two recombinants;

FIG. 6: segregation of the M1 marker in the HD population (IR64×Azucena); IR64-Azucena-30 HD individuals (IR64×Azucena);

FIG. 7: the genetic linkage map of chromosome 4 of rice with the positioning of marker M1 and identification of the space interval in which the resistance locus is found;

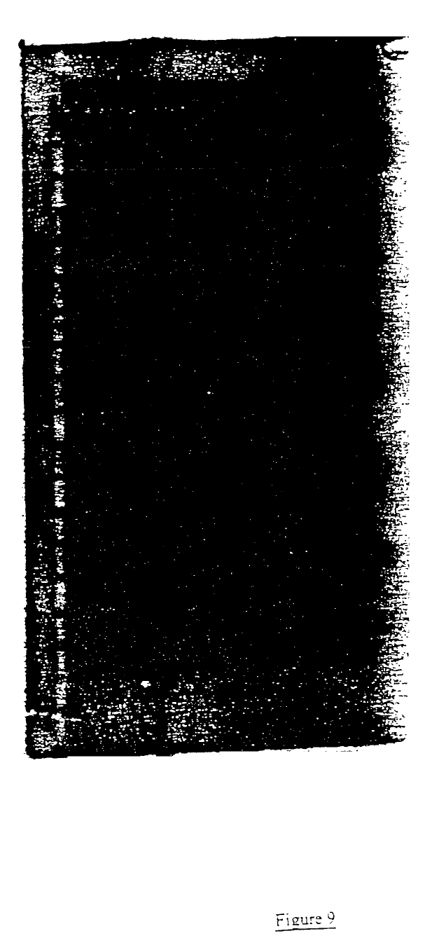

FIG. 8: hybridization of M1 marker used as probe on membranes carrying the DNA of the 4 varieties (IR64, Azucena, Gigante and Tog 5681) digested by 6 restriction enzymes ApaI, KpnI, PstI, Scal, HaeIII. The Tog5681 variety shows a different restriction profile to the other varieties for the Scal enzyme which may be used to label the resistance locus of this variety; and

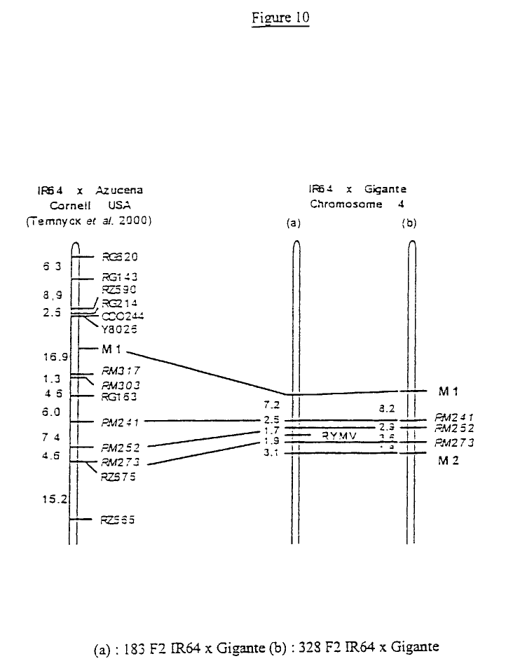

FIG. 9: hybridisation of the M1 marker used as probe on membranes carrying the DNA of individuals derived from backcross (IR64×Tog568) ×Tog 5681 and digested with the Scal enzyme. These descendants are in segregation for RYMV resistance. The sensitive individuals (5) all show the IR64 band associated with the Tog5681 band (heterozygote individuals). The resistant individuals (9) only show the Tog5681 band with the exception of one recombinant individual,

FIG. 10: mapping and anchoring of the locus of bred resistance to RYMV on the map IR64×Azucena, and

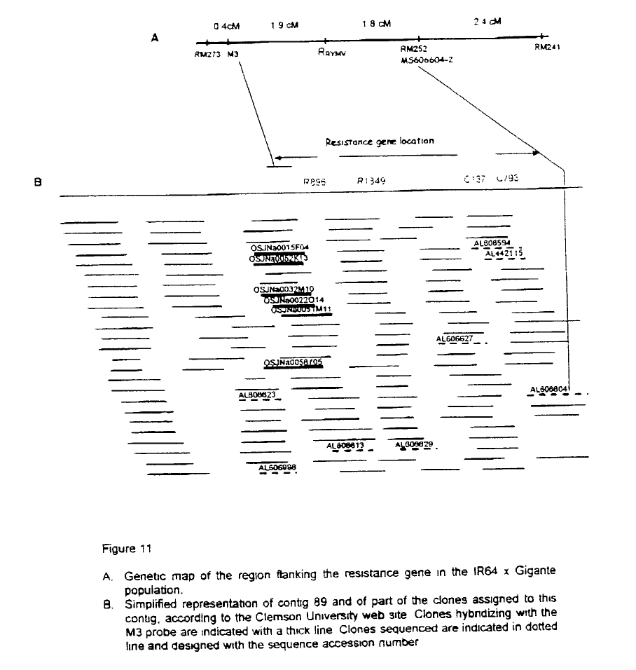

FIG. 11, the genetic map of the region flanking the resistance gene in the IR64×Gigante population (figure 11A) and the simplified representation of contig 89 and of part of the clones assigned to this contig.

### EXAMPLE 1

### Identification of resistant-source varieties

The varieties used in the resistance study, and especially the two resistant varieties Gigante and Tog5681, were characterized using microsatellite markers on a representative sampling of loci.

Polymorphism is evidenced by the number of repeats of a short nucleotide pattern, most often binucleotide which is characteristic of a given variety.

On a set of loci, the catalogued alleles can provide specific characteristics for each variety.

The detection of these microsatellite markers is made by DNA amplification using the specific primers determined by Chen et al (1) followed by migration on polyacrylamide gel under denaturing conditions in accordance with the protocol described by the same authors.

Table 1 gives the results using a reference system drawn up by Chen et al above, according to which the alleles are identified by the number of pattern repeats compared with the IR36 variety used as control. The two varieties Gigante and Tog5681 are therefore specifically described on 15 loci in respect of any other varieties (the microsatellite markers are given in column one).

TABLE 1

| | | Size on | | | | | | |
|---|---|---|---|---|---|---|---|---|
| Locus | Chr | IR36 | Ref. | IR36 | Gigante | IR64 | Azucena | Tog568113 |
| RM001 | 1 | 113 | (2) | n | n − 26 | n | n − 22 | n − 26 |
| RM005 | 1 | 113 | (2) | n | n − 6 | n − 4 | n + 16 | n − 8 |
| RM011 | 7 | 140 | (2) | n | n − 4 | n | n − 24 | n − 16 |
| RM018 | 7 | 157 | (2) | n | n + 4 | n + 6 | n + 8 | n − 6 |
| RM019 | 12 | 226 | (2) | n | n | n + 21 | n − 9 | n − 21 |
| RM021 | 11 | 157 | (2) | n | n + 8 | n | n − 14 | n − 32 |
| RM148 | 3 | 129 | (3) | n | n + 6 | n | n | n + 6 |
| RM167 | 11 | 128 | (3) | n | n + 4 | n | n + 32 | n + 24 |
| RM168 | 3 | 116 | (3) | n | n − 20 | n | n − 20 | n − 24 |
| RM232 | 3 | 158 | (1) | n | n − 14 | n | n − 12 | n − 16 |
| RM022 | 3 | 194 | (2) | n | n − 2 | n | n − 4 | n − 2 |
| RM252 | 4 | 216 | (1) | n | n + 38 | n + 2 | n − 20 | n + 10 |
| RM255 | 4 | 144 | (1) | n | n | n | n | n |
| RM246 | 1 | 116 | (1) | n | n − 12 | n − 12 | n − 16 | n − 12 |
| RM231 | 3 | 182 | (1) | n | n + 6 | n − 22 | n − 4 | n − 12 |

### EXAMPLE 2

### Characterization of resistance

Resistance was characterized using artificial inoculation of young seedlings with the virus, compared with an extremely sensitive control variety IR64.

The virus content was followed up for 60 days after inoculation using ELISA tests on the most recent leaves.

These tests were never able to demonstrate a signal that was significantly different to the signal of control plants non-inoculated with the virus.

A further experiment was conducted by inoculating isolated protoplasts of the two varieties Tog5681 and Gigante. In both cases, it was possible to detect the presence of viral proteins (capsid protein and P1 movement protein) and the accumulation of viral DNA, demonstrating the capacity of these protoplasts to multiply the virus, in the same manner as the protoplasts of sensitive varieties such as IR64.

Therefore, if it is considered that replication, cell-to-cell movement and long-distance transport through the vessels are the three main steps in the process of the infectious cycle within the plant, the resistance of these two varieties most logically lies in blockage of the virus at the infected cells.

### EXAMPLE 3

### Resistance genetics

Different F1 crosses were made between the resistant O. sativa variety (Gigante), a resistant O. glaberrima variety (Tog5681- also identified by ADRAO), and the highly sensitive control variety IR64 (selected at the IRRI).

Culture of the plant material, crosses and production of descendants were made in the IRD greenhouses in Montpellier.

The F1 hybrids obtained between the sensitive and resistant varieties were tested for resistance to the RYMV virus by ELISA testing and follow-up of symptoms.

These F1 hybrids proved to be as sensitive as the sensitive parent, and therefore showed that that the type of resistance is recessive.

On the other hand, the hybrids between the two resistance sources Gigante and Tog5681 only yielded resistant F1 hybrids to the benefit of a single resistance locus in these sources of resistance.

These results are summarized in Table 2 below.

This table gives the distribution of ELISA responses (A 405 nm) in the leaves infected by systemic route of Fl hybrids, of backcrosses and of F2 descendants obtained from backcrosses between the sensitive IR64 variety and the 2 resistant cultivars Gigante and Tog5681.

TABLE 2

| | | | | | | Average |
|---|---|---|---|---|---|---|
| | Presence | Number of | Distribution of OD values | | | values |
| F1 hybrid descendants | of symptoms | genotypes | (0.01-0.05) | (0.9-1) | >1 | OD |
| Derivatives of Tog5681 | | | | | | |
| F1: (IR64 × Tog 5681) | Sensitive | — | — | — | 10 | 1.9 |
| BCS: (IR64 × Tog 5681) × IR64 | Sensitive | 19 | 6 | 4 | 15 | 1.6 |
| BCS: (IR64 × Tog5681 c Tog5681 | In segregation | 22 | 12 | — | 10 | — |
| Derivatives of fertile BCS | | | | | | |
| plant | | | | | | |
| BCS F2 | Sensitive | 11 | — | — | 11 | 1.3 |
| BCS × IR64 | Sensitive | 1 | — | — | 1 | 1.9 |
| BCS × Tog5681 | sensitive | 15 | — | — | 15 | 1.9 |
| Gigante derivatives | | — | — | — | | |
| F1 (IR64 × Gigante) | | — | — | — | 10 | 1.9 |
| F2: (IR64 × Gigante) | In segregation | 65 | 15 | — | 50 | — |
| F1: (Gigante × Tog5681) | Sensitive | — | 10 | — | — | 0.3 |

The ELISA responses were obtained from:
i) 10 plants regenerated by cuttings for each F1 hybrid combination
ii) 1 plant regenerated for each backcross-derived interspecific genotype
iii) direct tests on young seedlings (inoculation at 10 days after germination and read-off at 7 days after inoculation) for F2 and fertile interspecific descendants

In respect of Gigante, the heredity of resistance was confirmed by a resistance test on 55 F3 families resulting from the cross between (IR64×Gigante) . The results are given in Table 3.

This table gives the segregation of RYMV resistance in F3 descendants (IR64×Gigante). Inoculation was made 10 to 17 days after germination with the Burkina Faso isolate and symptoms were followed up for 45 days after inoculation.

TABLE 3

| Classes of | Number of | Number of plants | | | |
|---|---|---|---|---|---|
| resistance | descendants | Total | Sensitive | Resistant | Incidence of resistant plants |
| Sensitive | 15 | 191 | 191 | 0 | 0 |
| In segregation | 30 | 343 | 262 | 01 | 0.24 |
| | | | | | 2 = 0.07 (3:1) |
| Resistant | 4 | 45 | 14 | 31 | 0.69 |
| Very resistant | 6 | 87 | 0 | 87 | 1 |
| Resistant* | 7 | 73 | 23 | 50 | 0.60 |
| Very resistant* | 4 | 56 | 0 | 56 | 1 |

*F3 descendants derived from resistant F2 plants analysed by ELISA tests

EXAMINATION OF THIS TABLE SHOWS THAT

¼ of F2 plants only give resistant plants in F3 descendants, and are homozygote for resistance,

¼ of F2 plants only give sensitive plants in F3 descendants, and are homozygote for sensitivity,

½ of F2 plants are in segregation for resistance and give sensitive and resistant plants in the same proportion (3:1) in F3 descendants.

All these results tally perfectly with a single recessive resistance gene occurring in the two varieties Gigante and Tog5681.

### EXAMPLE 4

### Identification M1 and M2 resistance markers using the AFLP protocol

A - OBTAINING DNA POOLS

The leaves of 10 sensitive plants and 10 resistant plants derived from an F2(IR64×Gigante) were sampled for their DNA extraction.

The DNA were then mixed stoechiometric fashion to form two DNA pools respectively corresponding to 10 sensitive or resistant F2 plants and with a final mixture concentration of 50 ng/μ l. These mixtures served as basis for the identification of resistance markers using the AFLP (Amplified Fragments Length Polymorphism) method developed by Zaneau et al (4) and Vos et al (5). The products used are in the form of a commercial kit (Gibco BRL) available from Keygene & Life Technologies.

b - Obtaining restriction fragments

250 ng of each of the DNA pools at 50 ng/μ l and of the parents are digested simultaneously by two restriction enzymes (EcoRI and MseI).

Digestion reaction (25 μl)

5 μl DNA (50 ng/ml)

0.2 μl (2U) EcoRI (10U/μl)

0.2 μl (2U) MseI (5U/μl)

5 μl 5X T4 ligase buffer

14.5 μl H₂ O

The digestion reaction is carried out for two hours at 37° C., then for 15 min at 70° C. to inactivate the restriction enzymes. After digestion, the ligation reaction was performed.

Ligation reaction (50 μl):

25 μl double digestion reaction medium

1 μl EcoRI adapter

1 μl MseI adapter

5 μl 5X T4 ligase buffer

1 μl (1 U) ligase (10U/μ l)

17 μl H₂ O

The ligation reaction is conducted at 37° C. for 3 hours followed by inactivation of the enzyme at 60° C. for 10 min.

c - Amplification

Amplification properly so-called was performed in two steps: preamplification and specific amplification.

c1- Preamplification reaction (50 μl)

5 μl of reaction medium containing the digested DNA fixed to the adapters, diluted to 1/10 0.5 μl EcoRI primer (150 ng/μl)

2 μl 5 mM nucleotide mixture

5 μl 10X buffer, Promega

5 μl MgCl₂, 25 mM

0.2 μl (1U) Taq polymerase (5U/μl)

31.8 μl H₂ O

The characteristics of PCR pre-amplification are the following:

20 cycles with

denaturing: 30 sec at 940° C.

hybridization: 30 sec at 56° C.

elongation: 1 min at 72° C.

Selective amplification is made using an aliquot of the first amplification diluted to 1/30 using primers having 3 selective nucleotides at the 3′ end, and by labelling one of the primers to develop bands on autoradiography film.

The following primer pairs are used:

E-AAC/M-CAG

E-ACC/M-CAG

E-AGC/M-CAG

in which

E meets the sequence:

GAC TGC GTA CCA ATT C (SEQ ID N^{o} 1), and

M meets the sequence:

GAT GAG TCC TGA GTA A (SEQ ID NO²)

The hybridization temperature is reduced by 0.7° C. per cycle, throughout the 11 following cycles:

last 20 cycles

denaturing: 30 sec at 90° C.

hybridization: 30 sec at 56° C.

elongation: 1 min at 72° C.

The EcoRI primer is labelled (for 0.5 μl tube):

0.18 μl EcoRI primer (5 ng)

0.1 μl γ³³ P ATP (10 mCu/μl)

0.05 μl 10X kinase buffer

0.02 μl (0.2u) T4 polymerase kinase (10U/μl)

0.15 μl H₂ O

The labelling reaction is conducted at 37° C. for 1 hour and is halted by 10 minutes at 70° C.

c2- Specific amplification reaction

(20 μl)

0.5 μl labelled EcoRI primer

5 μl preamplification reaction medium, diluted to

1/30

0.3 μl MseI primer (100 ng/μl)

0.8 μl 5 mM nucleotide mixture

2 μl 10X buffer, Promega

2 μl MgCl₂, 25 mM

0.1 μl (0.5U) Taq polymerase (5U/μl)

9.3 μl H₂ O a

Amplification characteristics are as follows:

32 cycles with

for the first cycle:

denaturing: 30 sec at 94° C.

hybridization: 30 sec at 65° C.

elongation: 1 min at 72° C.

for the 11 following cycles: the same conditions as previously, reducing the hybridization temperature by 0.7° C. for each cycle; and

for the 20 last cycles:

denaturing: 30 sec at 90° C.

hybridization: 30 sec at 56° C.

elongation: 1 min at 72° C.

d) Electrophoresis and Autoradiography

At the end of the amplification reaction, 20μl of charge buffer are added (98% formamide, 0.005% xylene cyanol and 0.005% bromophenol blue). The amplification products are separated by electrophoresis on denaturing polyacrylamide gel (6% acrylamide, 8 M urea) with a TBE migration buffer (18 mM Tris, 0.4 mM EDTA, 18 mM boric acid, pH 8.0) for 3 hours' migration at a power of 50 watts. After migration, the gel is fixed in a solution of 1 part acetic acid/ 2 parts absolute ethanol for 20 minutes. The gel is transferred to 3 M Wattman paper and dried for 45 minutes at 80° C. with a gel drier. The gel is placed in a cassette with ultrasensitive film. The autoradiograph is developed after two days' exposure. Comparison of the profiles obtained with the parents and the pools of sensitive of resistant plants led to identifying bands present in one of the pools but absent in the other. These bands, candidates for resistance marking, were then verified individually on each of the plants forming the DNA pools.

e) Results

Study of the results obtained shows that the two markers called M1 and M2 are present in the sensitive parent (IR64) and in all F2 plants (IR64×Gigante) forming the pool of sensitive plants, whereas this band is absent in the resistant parent (Gigante) and that only one individual in the resistant pool shows this band. The same type of variation is observed in backcross (IR64×Tog55681)×Tog 5681. The other markers identified by this analysis (M3 to M6) also show the same variation:

presence of bands in the sensitive parent and the pool of F2 sensitive plants (IR64×Gigante) and in the sensitive plants of the backcross (IR64×Tog5681) ×Tog5681).

absence of bands in the resistant parents Gigante and Tog5681, in the pool of F2 resistant plants (IR64×Gigante) and in the resistant plants of the backcross (IR64×Tog5681) ×Tog5681.

The segregation data between the AFLP markers M1 to M6, the resistance locus for the F2 pools (IR64×Gigante) and the interspecific backcross (IR64×Tog5681) ×Tog5681 are summarized in tables 4 and 5. Analysis of the segregation data and of the rare recombinants observed in both crosses can be used to assess the recombination rates between these different markers and the resistance locus. In particular, markers Ml firstly and markers M2 to M6 secondly determine a segment of less than 10-15 cM carrying the resistance locus. M1 and M2 are therefore less than 5-10 cM apart and are positioned either side of this locus.

TABLE 4

| Resistance/Marker M1 | N° of individuals observed | | | | | | |
|---|---|---|---|---|---|---|---|
| Phenotype | Resistant | | | Sensitive | | | |
| RYMV resistance genotype | tt/gg | tt | gg | It | It | It | It |
| AFLP marker | −/− | +/− | +/ | −/− | +/− | −/− | +/ |
| Resistant F2 pool | 10 | — | 1 | — | — | — | — |
| (IR64 × Gigante) | | | | | | | |
| Sensitive F2 pool | — | — | — | — | — | 0 | 10 |
| (IR64 × Gigante) | | | | | | | |
| Interspecific backcross | 11 | 1 | — | 0 | 8 | — | — |
| Tog5681 | | | | | | | |
| Resistance/Marker | | | | | | | |
| M2, M3, M4, M6 | N° of individuals observed | | | | | | |
| Phenotype | Resistant | | | Sensitive | | | |
| RYMV resistance genotype | tt/gg | tt | gg | It | It | II | II |
| AFLP marker | −/− | +/− | +/ | −/− | +/− | −/− | +/ |
| Resistant F2 pool | 11 | — | 0 | — | — | — | — |
| (IR64 × Gigante) | | | | | | | |
| Sensitive F2 pool | — | — | — | — | — | 0 | 10 |
| (IR64 × Gigante) | | | | | | | |
| Interspecific backcross | 10 | 2 | — | 0 | 8 | — | — |
| Tog5681 | | | | | | | |
| Resistance/Marker M5 | N° of individuals observed | | | | | | |
| Phenotype | Resistant | | | Sensitive | | | |
| RYMV resistance genotype | tt/gg | tt | gg | It | It | II | II |
| AFLP marker | −/ | +/− | +/ | −/− | +/− | −/− | +/ |
| Resistant F2 pool | 11 | — | — | — | — | — | 0 |
| (IR64 × Gigante) | | | | | | | |
| Sensitive F2 pool | — | — | — | — | — | 0 | 10 |
| (IR64 × Gigante) | | | | | | | |
| Interspecific backcross | 9 | 3 | 0 | 8 | — | — | — |
| Tog5681 | | | | | | | |

TABLE 5

| Marker M1/Markers M2, M3, M4, M6 | N° individuals observed | | | |
|---|---|---|---|---|
| Genotype M1 | −/* | +/* | −/− | −/− |
| Genotype M2, M3, M4, M6 | +/* | −/− | +/* | −/− |
| Resistant F2 pool | 0 | 1 | 0 | 10 |
| (IR64 × Gigante) | | | | |
| Sensitive F2 pool | 10 | 0 | 0 | 0 |
| (IR64 × Gigante) | | | | |
| Interspecific backcross | 11 | 2 | 2 | 11 |
| Tog5681 | | | | |
| Marker M1/Marker M5 | N° individuals observed | | | |
| Genotype M1 | −/* | +/* | −/− | −/− |
| Genotype M5 | +/* | −/− | +/* | −/− |
| Resistant F2 pool | 0 | 1 | 0 | 10 |
| (IR64 × Gigante) | | | | |
| Sensitive F2 pool | 10 | 0 | 0 | 0 |
| (IR64 × Gigante) | | | | |
| Interspecific backcross Tog5681 | 11 | 2 | 3 | 10 |
| Marker M5/Markers M2, M3, M4, M6 | N° individuals observed | | | |
| Genotyne M5 | +/* | +/* | −/− | −/− |
| Genotype M2, M3, M4, M6 | +/* | −/− | +/* | −/− |
| Resistant F2 pool | 0 | 0 | 0 | 11 |
| (IR64 × Gigante) | | | | |
| Sensitive F2 pool | 10 | 0 | 0 | 0 |
| (IR64 × Gigante) | | | | |
| Interspecific backcross Tog5681 | 13 | 1 | 0 | 12 |

*(−) interspecific backcross Tog5681 (+ or −) F2 pool.

### EXAMPLE 5

### ISOLATION OF MARKER M1

A further amplification with the same pair of primers was conducted, followed by migration on polyacrylamide gel under the same conditions as above. Development was carried out by staining with silver nitrate using the silver staining kit (Promega) for direct viewing of the bands on the gel. After development, the Ml band was excised from the gel, then the DNA was eluted in 50 μl water at 4° C. overnight.

An aliquot of 5 μl was taken and re-amplified using the same primer pairs with P³³ labelling. The amplification product was again separated on 6% denaturing acrylamide gel and compared with the parents and the sensitive and resistant pools. The lane corresponding to this amplification product shows a single band of 510 bp migrating at exactly the same level as the original band which had been excised. Another 5 μl aliquot was also amplified with the same primers and separated on 1.8% agarose gel. The band corresponding to the expected size (510 bp) was again excised and purified with a gene clean kit (Promega)

### EXAMPLE 6

### CLONING AND SEQUENCING OF THE M1 MARKER

cloning

3 μl of purification product was used for a cloning reaction overnight at 37° C.

3 μl purification product

1 μl PGEMTeasy vector

1 μl 10X T4 ligase buffer

1 μl T4 DNA Ligase

4 μl H₂ O

Transformation was conducted with the E.Coli strain JM109, adding 5 μl of cloning product to 100 μl competent E. coli JM109 cells. A pre-culture was made on LB culture medium for 1 hour at 37° C. The bacteria were subsequently spread over a Petri dish containing agar with 1/1000 ampicilline. 50 μl IPTG-XGal were added just before spreading the bacteria to select the transformed bacteria. A white colony ( transformed) was selected and replaced in culture under the same conditions (Agar plus ampicilline).

From this culture a miniprep of plasmid DNA was MADE using the Wizard Plus kit (Promega) . The plasmid DNA containing the insert was digested with the EcoRI enzyme to verify the presence of the Ml marker. 1.8% agarose gel was used to verify the presence of the 3 kb band corresponding to the plasmid and the 510 bp band corresponding to the M1 marker (photo 1).

Sequencing

The sequence of the insert (SEQ ID N^{o} 3) is the following (5′,3′):

SEQ ID N^{O}3

20 30 40 50 60 70
GTGCTTGCTTATAGCACTACAGGAGAAGGAAGGGGAACACAACAGCC
ATGGCGAGCGAAGGTTCAACGTCGGAGAAACAGGCTGCGACGGGCAG
CAAGGTGCCGGCGGCGGATCGGAGGAAGGAAAAGGAGGAAATCGAA
GTTATGCTGGAGCGGCTTGACCTAAGGGCAGATGAGGAGGAGGATGT
GGAATTGGAGGAAGATCTAGAGGAGCTTGAGGCAGATGCAAGATGGC
TAGCCCTAGCCACAGTTCATACGAAGCGATCGTTTAGTCAAGGGGCTT
TCTTTGGGAGTATGCGCTCAGCATGGAACTGCGCGAAAGAAGTAGATT
TCAGAGCAATGAAGACAATCTGTTCTCGATCCAATTCAATTGTTTGG
GGGATTGGAACGAGTTATGAATGAAGGTCCATGGACCTTTCGAGGAT
GTTCGGTGCTCCTCGCAGAATATGATGGCTGGTCCAAGATTGAAT

The sequences corresponding to the primers used for AFLP amplifications were found and show that the band corresponds to a restriction fragment (EcoRI-MseI).

By deducing the sequences corresponding to the primers, the actual size of the DNA fragment of the cloned rice is 471 bp.

The use of different pairs of primers (1-3), (1-4), (1-5) firstly and (2-3), (2-4), (2-5) secondly, makes it possible to validate the cloning of the AFLP M1 band. Amplification of the DNA of the varieties used in the crosses with these primers only shows one single band. The fragment corresponding to the Tog5681 variety is slightly larger than for the other varieties (FIG. 2).

### EXAMPLE 7

### TRANSFORMATION OF THE M1 SEQUENCE INTO A POLYMORPHOUS MARKER

A polymorphism for the Ml marker was determined between the parents of the doubled haploid population (IR64×Azucena) . This population totals over 300 markers distributed over the 12 rice chromosomes. On this account, we relied on the restriction sites of the M1 marker sequence determined on the IR64 parent (FIG. 3). The primers (1-3), (1-4) and (1-5) were used to amplify the DNA of the parents of crossed plants which was then digested by restriction enzymes. The restriction site HpaII/MspI releases a fragment of 86 bp when primer 1 is used. This site is absent in the Gigante and Azucena varieties (FIG. 4).

The marker was tested on the F2 individuals of the sensitive pool and resistant crossed pool (IR64×Gigante). All the resistant individuals have the profile of the Gigante variety (absence of the M1 AFLP marker associated with absence of the restriction site HpaII/MspI) with the exception of individual (5.11). The sensitive individuals show the HpaII/MspI restriction site in the homozygote state like the IR64 variety with the exception of two heterozygote individuals which are recombinant (FIG. 5).

The sequence of the M1 marker which can be amplified with specific primers indeed corresponds to the M1 AFLP marker. Digestion by the HpaII/MspI enzyme leads to distinguishing between the allele derived from the sensitive parent (IR64) and from the resistant parent (Gigante).

With these new data, it is possible to give back-up to the positioning of the resistance locus between markers M1 and M2 and to estimate the recombination rate at 0.065 ±0.045 for the distance between M1 and the resistance locus, and 0.11 ±0.047 for the distance between markers M1 and M2.

### EXAMPLE 8

### MAPPING OF THE M1 MARKER

Sixty individuals from the (IR64×Azucena) population were passed as marker M1: amplification with primers (1-3) and digestion with the HpaII/MspI enzyme, followed by separation of the fragments on 2.5 % agarose gel. Segregation of marker M1 shows no distortion (FIG. 6) . The results are used to map the Ml marker using mapping software (Mapmaker V3) which leads to positioning the M1 marker on chromosome 4 between the markers RG163 and RG 214(FIG. 7) . This space represents the zone in which the RYMV resistance locus is located.

### EXAMPLE 9

### MARKING THE RESISTANCE LOCUS OF THE TOG5681 VARIETY

The presence of the restriction site HpaII/MspI in the Tog5681 variety means that it is not possible to use the strategy in example 8 to verify that the Ml marker is also a marker of Tog5681 resistance derived from Tog5681. Therefore, the 4 varieties Azucena, Gigante, IR64 and Tog5681 were digested with 12 restriction enzymes (BamHI, Bg/II, DraI, EcoRI, EcoRV, HindlIl, Apal, KpnI, PstI, Scal, XbaI, HaeIII) to identify a restriction polymorphism using the DNA sequence of the Ml marker as probe. The Scal enzyme leads to identifying a polymorphism between IR64 and Tog5681(FIG. 8). This polymorphism was used to validate the M1 marker on a backcross (IR64×Tog5681) ×IR64 in segregation for resistance. 5 sensitive individuals of this backcross were tested and all showed the characteristic band of IR64. The 9 resistant individuals only show the Tog5681 band with the exception of only one which is recombinant (FIG. 9). The restriction polymorphism revealed by the Scal enzyme using the M1 marker as probe is therefore related to the resistance locus of Tog5681. There is coherence between genetic analysis and the identification of resistance markers for considering that the M1 marker indeed maps the same resistance locus in the two varieties Gigante and Tog5681.

### EXAMPLE 10

### CLONING AND SEQUENCING OF THE M2 MARKER INTO A SPECIFIC PCR-MARKER

The AFLP band obtained with the pair of primers E-ACC/M-CAG corresponding to the M2 band visible in the sensitive parent (IR64) and present in all the individuals forming the sensitive pool, was cloned using the same protocol as for marker M1. The sequence corresponding to this band was determined and 3 primers were defined (1 forward - 2 reverse) to allow conversion of this marker into a specific PCR marker. Sequence of the M2 marker (120 bp) (SEQ ID NO.⁹):

AATTCACCCC ATGCCCTAAG TTAGGACGTT CTCAGCTTAG
TGGTGTGGTA GCTTTTTCTA TTTTCCTAAG CACCCATTGA
AGTATTTTGC ATTGGAGGTG GCCTTAGGTT TGCCTCTGTTA
Primers:

| | | |
|---|---|---|
| AACCTAAGGCCACCTCCAAT: | (right) | (SEQ ID NO 10) |
| GCAAACCTAAGGCCACCTC: | (right) | (SEQ ID NO 11) |
| ATTCACCCCATGCCCTAAG: | (left) | (SEQ ID NO 12) |

The following conditions were used to amplify markers M1 and M2 simultaneously:

| | |
|---|---|
| 10 X buffer, Promega | 1.5 μl |
| MgCl₂ Promega | 1.5 μl |
| dNTP (5 mM) | 0.6 μl |
| M1-1 primer (10 mM) | 0.15 μl |
| M1-4 primer (10 mM) | 0.15 μl |
| M2-1 primer (10 mM) | 0.15 μl |
| M2-2 primer (10 mM) | 0.15 μl |
| H₂O | 7.74 μl |
| Taq Polymerase | 0.06 μl |
| DNA (5 ng/μl) | 3.00 μl |

PCR programme:

5 min at 94° C.

1 mn at 94° C.

30 s at 59° C.

1 mn at 72° C.

35 cycles

5 mn at 72° C.

10 mn at 4° C.

The M2 marker may be amplified alone at a hybridization temperature of 60.5° C., the other parameters remaining unchanged. Under these amplification conditions, the M2 marker appears to be a dominant marker characterized by band presence in the sensitive parent (IR64) and band absence in the Gigante parent.

### EXAMPLE 11

### Creation of a population of recombinant resistant plants between markers M1 and M2 to arrange within this space the candidate AFLP markers for resistance marking.

750 F2 individuals (IR64×Gigante) were artificially inoculated with the RYMV virus (BFl strain). The symptom-free plants were transplanted to a greenhouse, i.e. 188 individuals. Subsequently, additional analysis based on ELISA and descendant tests made it possible to eliminate a last fraction of 50 sensitive plants. The remaining 138 plants, homozygote for resistance, were systematically genotyped for both markers M1 and M2 as previously described. In this manner, 45 individuals were selected (38 recombinant relative to M1. 7 recombinant relative to M2) and 2 double recombinants. These recombinant individuals were used for arranging the AFLP markers in the space between M1 and M2. These results are summarized in Table 6 below:

TABLE 6
Selection of a recombinant F2 sub-population (IR64 x
Gigante) in the M1-M2 marker space

| | N° of | |
|---|---|---|
| Steps conducted: F2 (IR64 × Gigante) | plants | % |
| Inoculation of F2 plants (10 days after sowing) | 768 | |
| Greenhouse transplantation (5 weeks after | 188 | |
| inoculation) | | |
| Elimination of sensitive plants | 50 | |
| (symptom follow-up - Elisa test, descendant test) | | |
| Selection of homozygote resistant plants for the | 138 | 17.9 |
| bred resistance gene | | |
| Genotyping of selected individuals for markers M1 | | |
| and M2 | | |
| Recombinant plants relative to M1 | 36 | 18.8 |
| Recombinant plants relative to M1 and M2 | 2 | 1.4 |
| Recombinant plants relative to M2 | 7 | 5.1 |

### EXAMPLE 12

### Screening of AFLP markers to select new candidate markers for resistance

A total of 328 primer pairs EcoRI/MseI, each one defined by 3 nucleotides, was used following the protocol previously described. These primers are given in Table 7 below.

TABLE 7

| Combination | EcoRI | MesI | Combination | EcoRI | MseI | Combination | EcoRI | MseI |
|---|---|---|---|---|---|---|---|---|
| NO | primer primer | NO | primer primer | NO | primer | | | |
| 1 | AAC | CAA | 55 | ACA | CTG | 109 | ACG | AGG |
| 2 | AAC | CAC | 56 | ACA | CTT | 110 | ACG | ACT |
| 3* | AAC | CAG | 57 | ACA | AAC | 111 | ACT | CAA |
| 4 | AAC | CAT | 58 | ACA | AAG | 112 | ACT | CAC |
| 5 | AAC | CCA | 59 | ACA | AAT | 113 | ACT | CAC |
| 6 | AAC | CCT | 60 | ACA | ACA | 114 | ACT | CAT |
| 7 | AAC | CGA | 61 | ACA | ACG | 115 | ACT | CCA |
| 8 | AAC | CGT | 62 | ACA | ACG | 116 | ACT | CGT |
| 9 | AAC | CTA | 63 | ACA | ACT | 117 | ACT | CGA |
| 10 | AAC | CTC | 64 | ACA | AGC | 118 | ACT | CGT |
| 11 | AAC | CTG | 65 | ACA | AGG | 119 | ACT | CTA |
| 12 | AAC | CTT | 66 | ACA | AGT | 120 | ACT | CTC |
| 13 | AAC | AAC | 67 | ACC | CAA | 121 | ACT | CTG |
| 14 | AAC | AAG | 68 | ACC | CAC | 122 | ACT | CTT |
| 15 | AAC | AAT | 69* | ACC | CAG | 123 | ACT | AAC |
| 16 | AAC | ACA | 70 | ACC | CAT | 124 | ACT | AAG |
| 17 | AAC | ACC | 71 | ACC | CCA | 125 | ACT | AAT |
| 18 | AAC | ACG | 72 | ACC | CCT | 126 | ACT | ACA |
| 19 | AAC | ACT | 73 | ACC | CCA | 127 | ACT | ACC |
| 20 | AAC | AGC | 74 | ACC | CGT | 128 | ACT | ACG |
| 21 | AAC | ACG | 75 | ACC | CTA | 129 | ACT | ACT |
| 22 | AAC | ACT | 76 | ACC | CTC | 130 | ACT | AGC |
| 23 | AAC | CAA | 77** | ACC | CTG | 131 | ACT | AGG |
| 24 | AAG | CAC | 78 | ACC | CTT | 132 | ACT | AGT |
| 25 | AAG | CAG | 79 | ACC | AAC | 133 | AGA | CAA |
| 26 | AAG | CAT | 80 | ACC | AAG | 134 | AGA | CAC |
| 27 | AAG | CCA | 81** | ACC | AAT | 135 | AGA | CAG |
| 28 | AAG | CCT | 82 | ACC | ACA | 136 | ACA | CAT |
| 29 | AAG | CGA | 83 | ACC | ACC | 137 | AGA | CCA |
| 30 | AAG | CGT | 84 | ACC | AGG | 138 | AGA | CCT |
| 31 | AAG | CTA | 85 | ACC | ACT | 139 | AGA | CGA |
| 32 | AAC | CTC | 86** | ACC | AGC | 140 | AGA | CGT |
| 33 | AAG | CTG | 87 | ACC | AGG | 141 | AGA | CTA |
| 34 | AAC | CTT | 88 | ACC | AGT | 142 | AGA | CTC |
| 35 | AAG | AAC | 89 | ACG | CAA | 143 | ACA | CTG |
| 36 | AAG | AAG | 90 | ACG | CAC | 144 | ACA | CTT |
| 37 | AAG | AAT | 91** | ACG | GAG | 145 | AGA | AAC |
| 38 | AAG | ACA | 92 | ACG | CAT | 146 | AGA | AAG |
| 39 | AAG | ACC | 93 | ACG | CCA | 147 | AGA | AAT |
| 40 | AAG | ACG | 94 | ACG | CCT | 148 | AGA | ACA |
| 41 | AAC | ACT | 95 | ACC | CGA | 149 | AGA | ACC |
| 42 | AAG | AGC | 96 | ACG | CCT | 150 | ACA | ACG |
| 43 | AAG | ACG | 97 | ACG | CTA | 151 | AGA | ACT |
| 44 | AAG | AGT | 98 | ACG | CTC | 152 | ACA | ACC |
| 45 | ACA | CAA | 99 | ACG | CTC | 153 | AGA | AGG |
| 46 | ACA | CAC | 100 | ACG | CTT | 154*** | ACA | ACT |
| 47 | ACA | CAC | 101 | ACC | AAC | 155 | AGC | CAA |
| 48 | ACA | CAT | 102 | ACG | AAC | 156 | ACC | CAC |
| 49 | ACA | CCA | 103 | ACG | AAT | 157*** | ACC | CAG |
| 50 | ACA | CCT | 104* | ACG | ACA | 158 | AGC | CAT |
| 51 | ACA | CCA | 105 | ACG | ACC | 159 | AGC | CCA |
| 52 | ACA | CGT | 106 | ACG | ACG | 160 | AGC | CCT |
| 53 | ACA | CTA | 107 | ACG | ACT | 161 | ACC | CGA |
| 54 | ACA | CTC | 108 | ACG | ACC | 162 | ACC | CGT |

Shaded: polymorphism for one or more bands between the sensitive and re-
sistant pools
* presence of one or more polymorphous bands in sensitive pool
** presence of one or more polymorphous bands in resistant pool
*** presence of one or more polymorphous bands in sensitive pool and re-
sistant pool

| 163 | AGC | CTA | 218 | AGT | AGC | 273 | CAT | CTA |
|---|---|---|---|---|---|---|---|---|
| 164 | AGC | CTC | 219 | ACT | ACG | 274 | CAT | CTC |
| 165 | AGC | CTC | 220* | AGT | ACT | 275 | CAT | CTG |
| 166 | ACC | CTT | 221 | ATC | CAA | 276 | CAT | CTT |
| 167 | ACC | AAC | 222 | ATC | CAC | 277 | CAT | AAC |
| 168 | AGC | AAC | 223 | ATC | CAG | 278 | CAT | AAG |
| 169 | AGC | AAT | 224 | ATC | CAT | 279 | CAT | AAT |
| 170 | ACC | ACA | 225 | ATC | CCA | 280* | CAT | ACA |
| 171 | AGC | ACC | 226 | ATC | CCT | 281 | CAT | ACC |
| 172 | AGC | ACG | 227 | ATC | CGA | 282 | CAT | ACG |
| 173 | AGC | ACT | 228 | ATC | CGT | 283 | CAT | ACT |
| 174** | AGC | AGC | 229 | ATC | CTA | 284 | CAT | ACC |
| 175*** | AGC | AGG | 230 | ATC | CTC | 285 | CAT | AGG |
| 176 | AGC | ACT | 231 | ATC | CTG | 286 | CAT | AGT |
| 177 | AGC | CAA | 232 | ATC | CTT | 287* | ACT | CAA |
| 178 | AAC | CAC | 233*** | ATC | AAC | 288 | CTA | CAC |
| 179 | AGG | CAG | 234*** | ATC | AAG | 289 | CTA | CAG |
| 180 | AGG | CAT | 235* | ATC | AAT | 290 | CTA | CAT |
| 181 | AGG | CCA | 236 | ATC | ACA | 291* | CTA | CCA |
| 182 | AGG | CCT | 237 | ATC | ACC | 292 | CTA | CCT |
| 183 | AGG | CGA | 238 | ATC | ACG | 293 | CTA | CCA |
| 184 | AGG | CGT | 239 | ATC | ACT | 294 | CTA | CGT |
| 185 | AGG | CTA | 240 | ATC | AGC | 295 | CTA | CTA |
| 186 | AGG | CTC | 241 | ATC | AGG | 296 | CTA | CTC |
| 187 | AGG | CTG | 242 | ATC | ACT | 297* | CTA | CTG |
| 188 | AGG | CTT | 243 | CAA | CAA | 298 | CTA | CTT |
| 189 | AGG | AAC | 244 | CAA | CAC | 299 | CTA | AAC |
| 190 | AGG | AAG | 245 | CAA | CAG | 300 | CTA | AAG |
| 191 | AGG | AAT | 246 | CAA | CAT | 301 | CTA | AAT |
| 192 | AGG | ACA | 24i | CAA | CCA | 302 | CTA | ACA |
| 193 | AGG | ACC | 248 | CAA | CCT | 303 | CTA | ACC |
| 194 | AGG | ACG | 249 | CAA | CGA | 304 | CTA | ACG |
| 195** | AGG | ACT | 250** | CAA | CGT | 305 | CTA | ACT |
| 196 | AGG | AGC | 251 | CAA | CTA | 306 | CTA | AGC |
| 197*** | AGG | AGG | 252 | CAA | CTC | 307 | CTA | AGG |
| 198 | AGG | ACT | 253 | CAA | CTG | 308 | CTA | ACT |
| 199 | ACT | CAA | 254* | CAA | CTT | 309 | CTT | CAA |
| 200 | ACT | CAC | 255 | CAA | AAC | 310 | CTT | CAC |
| 201 | ACT | CAG | 256 | CAA | AAG | 311 | CTT | CAG |
| 202 | ACT | CAT | 257* | CAA | AAT | 312** | CTT | CAT |
| 203 | ACT | CCA | 258** | CAA | ACA | 313 | CTT | CCA |
| 204 | AGT | CCT | 259 | CAA | ACC | 314 | CTT | CCT |
| 205 | AGT | CGA | 260 | CAA | ACG | 315 | CTT | CGA |
| 206 | ACT | CGT | 261 | CAA | ACT | 316 | CTT | CGT |
| 207 | ACT | CTA | 262 | CAA | AGC | 32.7 | CTT | CTA |
| 208 | ACT | CTC | 263 | CAA | AGG | 318* | CTT | CTC |
| 209 | AGT | CTG | 264 | CAA | AGT | 319** | CTT | CTG |
| 210 | AGT | CTT | 265 | CAT | CAA | 320 | CTT | CTT |
| 211 | AGT | AAC | 266 | CAT | CAC | 321 | CTT | AAC |
| 212 | AGT | AAG | 267 | CAT | CAG | 322 | CTT | AAG |
| 213* | AGT | PAT | 268 | CAT | CAT | 323 | CTT | AAT |
| 214 | AGT | ACA | 269 | CAT | CCA | 324 | CTT | ACA |
| 215** | AGT | ACC | 270 | CAT | CCT | 325 | CTT | ACC |
| 216 | AGT | ACG | 271 | CAT | CGA | 326 | CTT | ACG |
| 217 | AGT | ACT | 272* | CAT | CGT | 327 | CTT | ACT |
| | | | | | | 328 | CTT | AGT |
| Shaded: polymorphism for one or more bands between the sensitive and resistant pools | | | | | | | | |
| * presence of one or more polymorphous bands in sensitive pool | | | | | | | | |
| ** presence of one or more polymorphous bands in resistant pool | | | | | | | | |
| *** presence of one or more polymorphous bands in sensitive pool and resistant pool | | | | | | | | |

With this screening, it was possible to identify one or more polymorphous bands according to their occurrence in the sensitive parent and/or resistant parent. 23 primer pairs were able to identify polymorphism between the parents confirmed by the F2 DNA pools, sensitive or resistant. The table below summarizes and gives the position in the M1-M2 space of AFLP markers bound to the locus of bred resistance the rice yellow mottle virus.

TABLE 8

| | |
|---|---|
| | Presence of |
| Variable | band(s) |

| | | | |
|---|---|---|---|
| Combi- | nucleotides | Sensi- | Resis- |

| | | | | | |
|---|---|---|---|---|---|
| nation | EcoRI | MseI | tive | tant | Marker position |
| Number | primer | primer | pool | pool | in M1-M2 space |

| | | | | | |
|---|---|---|---|---|---|
| 3 | AAC | CAG | − | − | =cloned M1 marker |
| 69 | ACC | CAG | + | − | =cloned M2 marker |
| 77 | ACC | CTG | − | + | non-determined |
| 81 | ACC | AAT | − | + | non-determined |
| 86 | ACC | AGC | − | + | non-determined |
| 91 | ACG | − | + | non-determined | |
| 104 | ACG | ACA | + | − | betw R and Rm273 |
| 154 | ACA | ACT | + | + | beyond M2 |
| 157 | AGC | CAG | − | + | in cosegr with M2 |
| 174 | AGC | AGC | − | + | non-determined |
| 175 | AGC | ACG | + | + | betw M1 and Rm241 |
| 197 | AGC | AGG | + | − | betw M1 and Rm241 |
| 215 | AGT | ACC | − | + | non-determined |
| 220 | ACT | AGT | + | − | betw Rm273 and M2 |
| 233 | ATC | AAG | + | + | betw M1 and Rm241 |
| 250 | CAA | CGT | − | + | non-determined |
| 254 | CAA | CTT | + | − | beyond M2 |
| 258 | CAA | ACA | + | − | betw M1 and Rm241 |
| 280 | CAT | ACA | + | − | beyond M2 |
| 287 | CTA | CAA | + | − | betw Rm273 and M2 |
| 291 | GTA | CCA | + | − | betw M1 and Rm241 |
| 318 | CTT | CTC | + | + | bewt Rm273 and M2 |
| 319 | CTT | CTG | − | + | non-determined |

After separate verification on each of the individuals forming the pools, the candidate markers corresponding to bands present in the IR64 parent may be tested on the recombinants identified in example 11. In this manner, 9 markers were confirmed as belonging to the M1-M2 space. Table 9 gives the order in the M1-M2 space of the AFLP markers identified by comparing sensitive and resistant DNA pools from a resistant F2 sub-ion (IR64×Gigante).

TABLE 9

| | | | | | | | | | | | | | | | |
|---|---|---|---|---|---|---|---|---|---|---|---|---|---|---|---|
| F2 | | | | | | | | | | | | | | | |
| Resistant | | E− | E− | E− | E− | E− | | | | E− | E− | C− | E− | | |
| individuals | | AGG | ATC | CAA | AGC | CTA | | | | ACG | | AGT | CTT | CTA | |
| (IR64× | | M− | M− | M− | M− | M− | | | RYMV | M− | | M− | M− | M− | |
| Gigante) | M1 | AGG | AGG | ACA | AGG | CCA | RM241 | RM252 | resist | ACA | RM273 | AGT | CTC | CAA | M2 |

| | | | | | | | | | | | | | | | |
|---|---|---|---|---|---|---|---|---|---|---|---|---|---|---|---|
| 2 | H | D | D | D | D | D | | B | B | B | B | B | B | B | B |
| 7 | H | D | D | D | D | D | | B | B | B | B | B | B | B | B |
| 8 | H | D | D | D | D | D | | B | B | B | B | B | B | B | B |
| 10 | H | D | D | D | E | D | | B | B | B | B | B | B | B | B |
| 21 | H | D | D | D | D | B | C | B | B | B | B | B | B | B | B |
| 23 | H | D | D | D | E | D | | B | B | B | B | B | B | B | B |
| 25 | H | D | D | D | D | D | E | H | B | B | B | B | B | B | |
| 28 | H | D | D | D | B | B | C | B | B | B | B | B | B | B | B |
| 37 | H | D | D | D | E | D | E | H | B | B | B | B | B | B | B |
| 48 | H | D | D | D | D | D | | B | B | B | B | B | B | B | B |
| 55 | H | D | D | D | D | D | E | H | B | B | B | B | B | B | B |
| 61 | H | D | D | D | D | D | E | H | B | B | B | B | B | B | B |
| 65 | H | D | D | D | | B | C | B | B | B | B | B | B | B | B |
| 95 | H | E | D | D | D | B | C | B | B | B | B | B | B | B | B |
| 103 | H | E | D | D | | B | C | B | B | B | B | B | B | B | B |
| 104 | H | D | D | D | B | B | C | B | B | B | B | B | B | B | B |
| 109 | H | D | D | D | D | D | C | B | B | B | B | B | B | B | B |
| 111 | H | E | D | D | D | D | | B | B | B | B | B | B | B | B |
| 119 | H | D | D | D | D | D | | B | B | B | B | B | B | B | B |
| 120 | A | D | D | D | D | B | C | B | B | B | B | B | B | B | B |
| 125 | H | E | E | E | E | D | | B | B | B | B | B | B | B | B |
| 127 | H | | | | | B | C | B | B | B | B | B | B | B | B |
| 131 | H | E | E | E | E | D | | B | B | B | B | B | B | B | B |
| 133 | H | | | | | B | C | B | B | B | B | B | B | B | B |
| 141 | H | E | E | E | E | D | E | H | B | B | B | B | B | B | B |
| 154 | H | E | E | E | E | D | | B | B | B | B | B | B | B | B |
| 158 | H | E | E | E | E | D | | B | B | B | B | B | B | B | B |
| 159 | H | | | | | B | C | B | B | B | B | B | B | B | B |
| 160 | H | E | E | E | E | D | | B | B | B | B | B | B | B | B |
| 151 | H | E | E | E | E | D | | B | B | B | B | B | B | B | B |
| 153 | H | | | | | B | C | B | B | B | B | B | B | B | B |
| 157 | H | | | | | B | B | B | B | B | B | B | B | B | B |
| 171 | H | | | | | B | B | B | B | B | B | B | B | B | B |
| 175 | H | E | E | E | D | D | B | B | B | B | B | B | B | B | B |
| 179 | H | | | | B | B | B | B | B | B | B | B | B | B | B |
| 183 | H | E | E | E | E | D | B | B | B | B | B | B | B | B | B |
| 35 | H | D | D | D | D | D | H | H | B | D | H | D | D | D | D |
| 135 | H | E | E | E | E | D | H | H | B | B | H | D | D | D | D |
| 17 | H | B | B | B | B | B | | B | B | D | H | D | D | D | D |
| 20 | B | B | B | B | B | B | B | B | B | D | H | D | D | D | D |
| 38 | B | B | B | B | B | B | | B | B | D | H | D | D | D | D |
| 93 | B | B | B | B | B | B | B | B | B | D | H | D | D | D | D |
| 105 | B | B | B | B | B | B | B | B | B | D | H | D | D | D | D |
| 145 | B | | | | B | B | B | B | B | B | B | B | B | B | D |
| 180 | B | | | B | B | B | B | B | B | B | B | D | D | D | D |

Incidence of recombinant individuals *

M1-R space 0.97 0.97 0.97 087 0.61 0.29 0.13

R-M2 space 0.67 0.78 0.89 0.89 0.89

Distance/resistance (cM) 11.4** 11.03 11.03 11.03 9.88 6.90 3.33 2.10 0.00 3.33 3.89 4.44 4.44 4.44 5.0**

A: genotype homozygote for the allele of the sensitive parent (IR64)

H: heterozygote genotype

B: homozygote genotype for the allele of the resistant parent (Gigante)

D: genotype non homozygote for the allele of the resistant parent (Gigante)

under the assumption of absence of double combination in space M1-R and M2-R

estimated distance using resistance map on 183 F2 (IR64×Gigante) cf (figure X)

14 bands from the resistant parent were also identified and will or will not be confirmed on recombinants generated in the F2 population (IR64×Gigante).

### EXAMPLE 13

### Anchoring of the RYMV resistance locus using microsatellite markers

The M1 marker being positioned on chromosome 4 of the genetic map (IR64×Azucena; example 9) microsatellite markers such as defined in (6) and belonging to this chromosome were used to fine-tune the map of the RYMV resistance locus. The following microsatellite markers were tested: RM241, RM252 (1), RM273 and RM177(6), under the experimental conditions described in (1) and (6). With the exception of the RM177 marker, non-polymorphous between the IR64 and Gigante parents, the markers RM241, RM252, RM273 were mapped on a F2 population (IR64×Gigante) assessed in parallel for RYMV resistance. The results on 183 F2 individuals make it possible to characterized a stretch of approximately 3.6 cM bordered by the two microsatellite loci RM252 and RM272 surrounding the RYMV resistance gene (see FIG. 10(a)).

### EXAMPLE 14

### Fine mapping of the space carrying the resistance locus and order of the resistance markers in the M1-M2 space.

The 45 F2 individuals (IR64×Gigante) resistant and recombinant for the M1 and m2 markers were characterized for the microsatellite markers identified in example 13. The mapping of the markers in segregation on all the F2 individuals (IR64×Gigante) available (321) confirms the order and the distance between the markers of the M1-M2 space, in particular the RM252-RM273 space which is estimated at 3.6 cM (FIG. 10(b)). With the 45 F2 individuals (IR64×Gigante) that are resistant and recombinant for the M1 and M2 markers, it is possible to confirm the order of the AFLP markers identified in example 12. One AFLP marker, EACG/MACA, remains within the RM252-RM273 space and represents the nearest marker to the RYMV resistance locus (Table 9). Overall, out of the 321 F2 individuals tested, there are 20 individuals recombined on one side or other of the RYMV resistance locus and may advantageously be used to identify closer markers and/or for cloning the resistance gene.

### EXAMPLE 15

### Marker-assisted resistance transfer

The markers close to the resistance locus were tested on irrigated varieties highly sensitive to the RYMV virus (var BG90-2, Bouake189, Jaya). 3 markers (M1, RM241, RM252) show polymorphism between these 3 varieties and the Gigante variety, enabling the use of these markers to be considered for resistance transfer to sensitive genotypes. Experimental transfer of resistance to these varieties was made as far as the 2^{nd} backcross. At each cross, the plants were verified for the presence of markers derived from Gigante, and resistance segregation was controlled by descendant tests on F2. Table 10 below summarizes results.

TABLE 10

| | | | | | | | theoretical | |
|---|---|---|---|---|---|---|---|---|
| | Polymorphism/donor parent | | | | | | % | N° of |
| Recurrent | (Gigante) | | | | | Generation | recurrent | lines |
| parent | M1 | RM241 | RM252 | RM273 | RM177 | obtained | parent | obtained |
| BG90-2 | poly | poly | poly | — | — | BC2F2 | 87.5 | 4 |
| Bouaké | poly | poly | poly | — | — | BC2F2 | 87.5 | 1 |
| 189 | poly | poly | poly | — | — | BC2F2 | 87.5 | 2 |
| Jaya | poly | poly | poly | poly | mono | BC3 | 93.7 | 5 |
| IR64 | | | | | | | | |

### EXAMPLE 16

### Anchoring of the resistance gene on the physical map

The AFLP band corresponding to the M3 marker and amplified in the susceptible parent IR64 with the primers E-ACG/M-ACA has been cloned using the same conditions than for Ml marker. This band was sequenced:

Sequence of M3 marker (excluded adaptators): Acggacctatccacttttatgccagcaagaaaatttagatgatggcaactgtatg t (seq. N^{o} 13)

DNA from varieties Gigante, IR64, Azucena and Tog5681 was digested using restriction enzymes Hind III, Eco RV, Dra I, Xba I, Bgl II, Bam HI, Sca I et Eco RI and membranes has been realized. Hybridization of the M3 sequence on these membranes did not reveal polymorphism between tested varieties. However, hybridization profile revealed that M3 is a single copy sequence in rice genome. This probe has been used to screen a BAC library including 36000 clones, realized in Clemson University using DNA of Nipponbare variety, digested with Hind III enzyme.

Membrane prehybridization was performed one night at 65° C. in hybridization tubes, in a buffer made of SDS 7%, sodium phosphate 0.5M pH7.2, EDTA 1 mM, salmon sperm DNA (0.1 mg/ml). Hybridization was performed in the same buffer in which labeled probe was added. Probe was radioactively labeled using the “5′-end-labelling” kit from Amersham-Pharmacia, as recommended by furnisher. After one night at 65° C., membranes were washed twice 20 minutes in SSC 1 X, SDS 0.1% and twice 20 minutes in SSC 0.5X, SDS 0.1%. The, membranes were wrapped in Saran-wrap and kept at −80° C. in contact with film.

Probe corresponding to M3 marker hybridized on 17 clones, 13 of which belong to contig 89, as described on Clemson University web site ( ) on JUN. 12, 2001. These clones were : OSJNBa0006L19, OSJNBa0015F04, OSJNBa0022014, OSJNBa0032M10, OSJNB a0048E10, OSJNBa0043I12, OSJNBa0051M11, OSJNBa0052K13, OSJNBa0059I01, OSJNBa0058F05, OSJNBa0070I17, OSJNBa0083D09. OSJNBa0087J22. These results are coherent enough to consider that M3 is on contig 89. A figure of the contig 89 is given (FIG. 11), clones hybridizing with M3 are indicated using a thick trait.

### EXAMPLE 17

### Position of the gene on contig 89

Several BAC of contig 89 have been sequenced in the international rice genome sequencing project and sequenced were released on data banks. Thus, the BAC clone OSJNBa0014 K14, localized, at one extremity of contig 89, has been sequenced and its sequenced has been recorded under accession number AL606604 on GenBank. A microsatellite sequence was identified on this clone and primers have been designed on both sides of this sequence in order to develop a microsatellite marker (referred later as MS606604-2).

Microsatellite sequence (upper case) and primers designed in flanking sequences (underlined):

GcaaagtgtttcaccttggacccatgcattCCTCCTCTCTCTCTCTCTCT
CTCTCTCTCTCTCTCTCTCTCTCTCTCTCTCTCTCTCTCTCTCTCTCTCT
CTCTCTCTCTCTCTCTCTCTCTCTCgagcgctcaactctccattgagcac
tgagcaggcccttacctttgcct

| | |
|---|---|
| Gcaaagtgtttcaccttggacc | (sequence NB 14) |
| Agcaggcccttacctttgcct | (sequence NB 15) |

This marker was amplified on varieties IR64, Gigante, Nipponbare and Tog5681 in the following conditions:

| | |
|---|---|
| dNTP | 200 μM |
| Taq | 0.02 U/μl |
| Buffer | 1X |
| MgCl2 | 1.5 mM |
| Forward primer-M13 | 0.1 μM |
| Reverse primer | 0.1 μM |
| Primer M13-IRD700 | 0.06 μM |
| (amplification in 15 μl) | |

In order to visualize amplification products on a LICOR sequencer, amplification is performed using the M13-forward universal primer labeled with IRD700 and the forward MS606604-2 primer to which the sequence 13-forward is added in 5′ position (tailing protocol described by furnisher). Amplification is realized with the program:

5 min 94° C.

30 s 94° C.

30 s 57° C.

5 40 s 72° C.

(34 cycles)

5 min 72° C.

A size-based polymorphism was detected between IR64 and Gigante varieties. This marker has been tested on 30 individuals recombined between RM252 and RM273 (12 resistant plants already presented in table 9 and 18 additional individuals evaluated for resistance level on F3 progenies). The marker MS606604-2 showed a perfect co-segregation with RM252 (table 11)

TABLE 11

| | | | | | | | |
|---|---|---|---|---|---|---|---|
| | | | | RYMV | | | |
| M1 | RM241 | MS606604-2 | RM252 | resistance | M7 | RM273 | M2 |

Resistant F2 plants recombined between RM252 and RM273

| | | | | | | | | |
|---|---|---|---|---|---|---|---|---|
| F2-R17 | B | — | B | B | B | D | H | D |
| F2-R20 | B | B | B | B | B | D | H | D |
| F2-R25 | H | — | | H | B | B | B | B |
| F2-R36 | H | H | H | H | B | D | H | D |
| F2-R37 | H | — | H | H | B | B | B | B |
| F2-R38 | B | — | B | B | B | D | H | D |
| F2-R55 | H | — | | H | B | B | B | B |
| F2-R61 | H | — | H | H | B | B | B | B |
| F2-R93 | B | B | B | B | B | D | H | D |
| F2- | B | B | B | B | B | D | H | D |
| R105 | | | | | | | | |
| F2- | H | H | H | H | B | B | H | D |
| R135 | | | | | | | | |
| F2- | H | — | H | H | B | B | B | B |
| R141 | | | | | | | | |

F2 plants recombined between RM252 and RM273, and
evaluated for resistance on F3 progenies

| | | | | | | | | |
|---|---|---|---|---|---|---|---|---|
| BR5 (11) | H | H | | H | B | B | B | B |
| F2-1 | H | H | H | H | H | B | B | B |
| F2-16 | H | H | A | A | A | D | H | D |
| F2-19 | H | B | B | B | H | — | H | D |
| F2-95 | A | A | A | A | H | D | H | D |
| F2-113 | H | — | H | H | H | B | B | B |
| F2-114 | — | H | H | H | B | — | H | |
| F2-133 | H | A | A | A | H | D | H | D |
| F2-142 | H | — | H | H | B | B | B | B |
| F2-163 | B | — | B | B | B | D | H | D |
| F2-167 | B | — | H | H | H | D | A | D |
| F2-176 | A | A | A | A | A | D | H | D |
| F2-184 | — | — | H | H | H | D | A | D |
| F2-189 | H | — | H | H | H | D | B | B |
| F2-206 | — | H | H | H | H | B | B | |
| F2-223 | — | A | A | A | H | D | H | |
| F2-278 | B | — | B | B | B | — | H | D |
| F2-280 | H | A | A | A | A | D | H | D |
| F2-285 | — | — | | A | H | — | H | |

A genotype homozygous for the allele from susceptible parent (IR64)
H: genotype heterozygous
B: genotype homozygous for the allele from the resistant parent (Gigante)
D: genotype not homozygous for the allele from the resistant parent (Gigante)

Resistance gene is localized between markers M3 and MS606604-2 and thus between the position delimited by these markers on contig 89, as mentioned on FIG. 11.

### BIBLIOGRAPHIC REFERENCES

(1) Chen, X et al., (1997), Development of a microsatellite framework map providing genome-wide coverage in rice (Oryza saliva L) Theor Appl Genet 95: 553-567.

(2) Panaud, 0. et al., (1996), Development of microsatellite markers and characterization of simple sequence length polymorphism (SSLP) in rice (Oryza sativa L) Mol Gen Genet 252: 597-607.

(3) Wu K.S. et al., (1993), Abundance, polymorphism and genetic mapping of microsatellites in rice. Mol Gen Genet 241: 225-235.

(4) Zabeau et al., (1993), Selective restriction fragment amplification: a general method for DNA fingerprinting. EP 92402629.7.

(5) Vos et al., (1995), AFLP, a new technique for DNA fingerprinting. Nucleic Acids research 23: 4407-4414.

(6) Temnyck et al., (2000), Theor Appl Genet 100:697-712.

The entire content of all reference cited or referred to herein is incorporated herein by reference.

1. Method for identifying markers of the locus of a major resistance gene to RYMV, comprising:
selective amplification of rice DNA fragments firstly from resistant individuals, and secondly from sensitive individuals, descending from parental varieties, these fragments being previously subjected to a digestion step, then a ligation step to fix complementary primer adapters having at their end one or more specific nucleotides, one the primers of the pair being labelled for development purposes,
separation of the amplification products, by gel electrophoresis under denaturing conditions, and
comparison of the electrophoresis profiles obtained with mixtures of fragments derived from resistant descendants and mixtures derived from sensitive descendants, with fragments derived from parental varieties, for the purpose of identifying bands whose polymorphism is genetically linked to the resistance locus, this identification optionally being followed, for validation purposes, by verification on each individual and calculation of the genetic recombination rate between the marker and the resistance locus.

2. Method according to claim 1, characterized in that the DNA fragments are obtained by digestion of the genomic DNA of resistant plants and of sensitive plants, and their parents, using restriction enzymes.

3. Method according to claim 2, characterized in that as restriction enzymes EcoRI and MseI are used.

4. Method according to claim 2 or 3, characterized in that the restriction fragments are subjected to ligation to fix adapters.

5. Method according to claim 4, characterized in that the fragments obtained are amplified using primer pairs complementary to the adapters whose sequences are respectively GAC TGC GTA CCA ATT C(SEQ ID N^{o} 1) and GAT GAG TCC TGA GTA A(SEQ ID N^{o} 2).

6. Method according to claim 4 or 5, characterized in that the fragments obtained are amplified using primer pairs having at their end the respective motifs AAC and CAG, ACC and CAG or further AGC and CAG.

7. Method according to any of claims 1 to 6, characterized by the identification of resistance marker bands, M1 and M2, whose size is respectively 510 bp and 140 bp, such as determined by gel electrophoresis under denaturing conditions.

8. Method according to claim 7, characterized in that said marker bands determine a segment of less than 10-15 cM carrying the resistance locus.

9. Method according to claim 8, characterized in that said marker bands are located either side of the locus at less than 5-10 cM.

10. Method according to any of claims 1 to 9, characterized in that it also comprises an isolation step to isolate the identified marker bands.

11. Method according to claim 10, characterized by purification of the isolated marker bands in order to obtain DNA fragments.

12. Method according to claim 11, characterized by cloning of the marker bands into a vector and insertion of the vector in a host cell.

13. Method according to either of claims 11 or 12, characterized by the recovery and sequencing of the purified, cloned DNA fragments.

14. Method for obtaining markers having high specificity for the locus of a major RYMV resistance gene, characterized in that PCR primer pairs are defined complementary to the sequence of the cloned fragment, specific amplification of this fragment is carried out using these primer pairs, then the amplification products are subjected to migration on electrophoresis gel with or without previous digestion by a restriction enzyme to identify a polymorphism.

15. Polymorphous AFLP bands such as identified by the method according to any of claims 1 to 14 using rice plant DNA.

16. AFLP bands according to claim 15, characterized in that they are specifically evidenced in a RYMV-sensitive variety, and in the fraction of sensitive plants derived from crossing of this variety with a resistant variety.

17. DNA sequences corresponding to polymorphous bands according to claim 15 or 16, which can be used to define a segment of chromosome 4 of 10-15 cM carrying the RYMV resistance locus.

18. DNA sequences according to claim 17, characterized in that they correspond to EcoRI-MseI fragments.

19. DNA sequences according to claim 18, characterized by a respective size of 510 bp and 140 bp determined by gel electrophoresis under denaturing conditions.

20. DNA sequences according to any of claims 17 to 19, characterized in that they correspond to sequences flanking the resistance locus and located either side of the latter at 5-10 cM or even at less than 5 cM.

21. DNA sequence, characterized in that it meets SEQ ID N^{o} 3.

22. DNA sequence, characterized in that it meets SEQ ID N^{o} 9.

23. Cloning vectors, characterized in that they contain sequence SEQ ID N^{o} 3 according to claim 21 or sequence SEQ ID N^{o} 9 according to claim 22.

24. Host cells, characterized in that they are transformed by vectors according to claim 22.

25. Use of polymorphous bands according to claim 15 or 16 or of DNA sequences according to any of claims 17 to 22 for the identification of resistant phenotypes and transfer of the resistance gene.

26. Fragments of no more than 4-5cM of chromosome 4 and polymorphous AFLP bands according to claim 15 or 16 defining a segment of 4-5cM or less carrying the RYMV resistance locus.

27. Use of SEQ ID NO.³ or SEQ ID NO.⁹ or of microsatellite markers such as RM252 and RM273, or any other marker such as SEQ ID NO.¹³ of contig 89 of Nipponbare BAC library, and showing polymorphism between a sensitive variety and a resistant variety, to transfer resistance into a sensitive variety by marker-assisted selection.

28. Use of sequences of contig 89 of Nipponbare library for identifying the sequences of the gene responsible for resistance to the rice yellow mottle virus.
